(19) United States
(12) Patent Application Publication
(10) Pub. No.: US 2003/0124633 A1
(43) Pub. Date: Jul. 3, 2003

(54) Diagnostic methods

(76) Inventors: Vesa Kleimola, Turku (FI); Erkki Eerola, Turku (FI); Markku Viander, Turku (FI)

Correspondence Address: Ronald L. Grudziecki, BURNS, DOANE, SWECKER & MATHIS, L.L.P., P.O. Box 1404, Alexandria, VA, 22313-1404, US

(21) Appl. No.: 10/164,629

(22) Filed: Jun. 10, 2002

(30) Foreign Application Priority Data
Dec. 31, 2001 (FI) 20012608

(51) Int. Cl. G01N 33/554; G01N 33/569

(52) U.S. Cl. 435/7.32

(57) ABSTRACT

The present invention relates to the diagnosis of Helicobacter pylori infection. Specifically, the present invention relates to the use of an urine sample in a diagnostic method for the detection of Helicobacter pylori antigens or metabolites.

### FIELD OF THE INVENTION

The present invention relates to the diagnosis of Helicobacter pylori infection. Specifically, the present invention relates to the use of an urine sample in a diagnostic method for the detection of Helicobacter pylori antigens or metabolites.

### BACKGROUND OF THE INVENTION

Helicobacter pylori is a curved gram-negative bacterium found in the upper gastrointestinal tract of humans. Since the first isolation of the bacterium in 1982, a huge amount of evidence has accumulated on the association of H. pylori with various gastric disorders, including dyspepsia (heartburn, bloating and nausea), symptomatic or asymptomatic inflammation of gastric mucosa manifested as chronic superficial gastritis or chronic active gastritis, peptic ulcers of the stomach and duodenum, and even gastric cancer and various gastric lymphomas [Dunn, B. E., et al., Clinical Microbiology Reviews 10 (1997) 720-740]. At present, it is believed that nearly all cases of peptic ulcers formerly thought to be idiopathic are actually caused by H. pylori infection [NIH Concensus Conference, JAMA 276 (1994) 1710].

H. pylori is a world wide human pathogen. The other known species carrying the bacterium is nonhuman primates. H. pylori infections have been connected to the socio-economic development: in developing countries 70 to 90% of the population carries the bacterium, whereas in developed countries the prevalence of the infection is approximately 25 to 50%. The infection is acquired in childhood, usually before the age of 10 years and is believed that the rate of the incidence decreases with improved hygiene. However, the route of transmittance of the infection is not definitely known, although faecal-oral and oral-oral routes are thought to be most important (Dunn, B. E., et al., supra).

Various methods and assays, both invasive and non-invasive, are available for the diagnosis of H. pylori infection. The invasive methods involve gastric or duodenal biopsies. The biopsy samples can be examined visually or histologically, cultured for the bacteria, tested for the urease enzyme produced by H. pylori, or analysed with gene technology. Commercial products are available for most of these methods. Non-invasive methods include serological tests for the detection of antibodies to H. pylori and urea breath test using ¹³C or ¹⁴C-labelled urea, for both of which multiple commercial tests are available. Additionally, assays measuring substrate metabolism of H. pylori in serum [Moulton-Barret, R. G., et al., Am. J. Gastroenterol 88 (1993) 369-374] and in urine [Pathak, C. M., et al., Am. J. Gastroenterol 89 (1993) 734-738] have been described.

Immunoassays measure the presence of IgG, IgA or IgM antibodies against H. pylori in patients' serum or blood samples (see, for example, U.S. Pat. No. 5,262,156; Pyloriset EIA-A and EIA-G, Orion Diagnostica, Finland), urine samples (see, for example, U.S. Pat. No. 5,262,156), and saliva or other mucous secretion specimen (see, for example, U.S. Pat. No. 6,068,985; Home Helicobacter Test, Ani Biotech Oy, Finland). The determination of antibodies against H. pylori suffer from several drawbacks, such as the strong dependence of the antigen preparation which is used to capture the antibodies, cross reactions of antibodies from related bacterial species, and the relatively long time needed for reliable test results. The accuracy of the so-called “office-based” or “near-patient” tests offered for use in doctor's offices is poorer than that of conventional laboratory assays. [Cohen, H., et al., Gastroenterology 110 (1996) A83; Sadowski, D., et al., Gastroenterology 110 (1996) A246]. Importantly, these assays relying on the detection of specific antibodies against H. pylori are less suitable for use in the evaluation and follow-up of the treatment and cure, since the elevated antibody levels maintain for a long period of time after the treatment and cure of the infection. Follow-up studies show great variation in the decline of the antibody levels after treatment [Kosunen, T. U., et al., Lancet 339 (1992) 893-895; Cutler, A., et al., Dig. Dis. Sci. 38 (1993) 2262-2266], but usually several months are needed for a decline, which reliably predicts the cure.

The detection of H. pylori antigens or metabolites instead of specific antibodies against H. pylori in a biological sample addresses this drawback. U.S. Pat. Nos. 5,716,791, 5,871,942 and 5,932,430 disclose, inter alia, methods for the detection of H. pylori antigens in faecal samples by complexing the antigen with a polyclonal antibody and detecting the complex thus formed by a second antibody. International patent application WO01/44815 discloses the detection of H. pylori antigens in a blood samples with, for instance, an ELISA method. These methods are suggested for the follow-up of the effect of the treatment of H. pylori infection.

The sample, especially a faecal sample, may represent a problem. Many patients find the collection of one faecal sample, let alone the collection of several faecal samples necessary for the follow-up, unpleasant and not hygienic, and their compliance to the treatment may suffer. Similarly, the laboratory personnel may dislike the handling of the faecal specimen and the preparation of samples for such assays due to the inherent infection risk. Also the preparation of serum or faecal samples for the analysis takes time, which often is limited during the patient's visit at the hospital or the doctor's office.

The use of urine samples, which are easy to collect and ready for the analysis, would save time and effort both from the patient and the medical personnel. However, until now H. pylori antigens or metabolites have not been demonstrated in urine samples.

One object of the present invention is to provide novel alternatives to the diagnosis of H. pylori infection and to the detection of H. pylori antigens and/or metabolites produced by the bacterium.

Another object of the present invention is to provide alternative methods for a reliable follow-up of the effect of pharmacotherapy in combating H. pylori infection and for the ascertainment of the cure of the patient with minor inconvenience to the patient.

A further object of the present invention is to provide alternative methods for the detection of H. pylori infection, the methods being as reliably applicable to the use in doctor's offices and in heath care centres and in clinical laboratories.

### SUMMARY OF THE INVENTION

The present invention relates to the use of urine samples in the diagnosis of Helicobacter pylori infection and for the detection of the presence or absence of a H. pylori antigen or a metabolite produced by the bacterium.

The present invention also relates to a non-invasive method for the diagnosis of Helicobacter pylori infection the method comprising detecting the presence or absence of a Helicobacter pylori antigen or a metabolite produced by the bacterium in a urine sample.

### DESCRIPTION OF THE DRAWINGS

FIG. 1 shows the SPR binding isotherms of the detection of H. pylori in urine samples. Samples 1 and 3 were obtained from patients positive for Helicobacter pylori and sample 2 was obtained from a patient negative for Helicobacter pylori. “ref” indicates the blank and BSU indicates patient urine sample.

FIG. 2 shows the results of a sandwich ELISA performed with urine samples.

### DETAILED DESCRIPTION OF THE INVENTION

The present invention is based on the surprising observation that, contrary to what has been thought and shown previously, the Helicobacter pylori antigens and/or metabolites produced by the bacterium are stable and detectable in urine samples obtainable non-invasively from patients suffering from H. pylori infection.

Urinary antigens derived from Helicobacter pylori could be demonstrated employing the sensitive surface plasmon resonance (SPR) technique with a specific and sensitive carrier substrate in the biosensor. Specifically, the carrier substrate, used in the SPR measurement, contained Fab′-fragments of specific monoclonal anti-H. pylori antibodies directly attached on to the solid surface of carrier substrate together with N-[tris(hydroxymethyl)methyl]acrylamide used to prevent non-specific binding. For the preparation of the carrier substrate, Fab′-fragments of specific monoclonal anti-H pylori antibodies were attached onto a solid surface of an SPR device. Glass slides coated with a thin film of titanium, and with a thin film of gold were attached to a SPR prism surface plasmon resonance device. The flow cell was assembled on the prism. The Fab′-fragments were added into the flow cell and were allowed to interact with the gold-coated surface. After washing N[tris(hydroxy-methyl)methyl]acrylamide was allowed to interact with the surface. The surface was then blocked with bovine serum albumin (BSA).

When urine samples obtained from patiens suffering from H. pylori infection were contacted with the surface of the carrier substrate, an increase in the SPR signal was obtained indicating the presence of H. pyroli antigens.

Urinary antigens derived from Helicobacter pylori could also be demonstrated employing a sandwich ELISA. The principle of the assay was conventional. The wells of a microtiter plate were coated with antibodies specific for H. pylori antigens. Urine samples as well as alkaline phosphatase (AP) labelled antibodies were added to the wells in one step. During the incubation, H. pylori antigens present in the sample bind to the antibodies immobilized on the microtiter plate and to the AP labelled antibody conjugates thus forming a sandwich complex. The wells were washed in order to remove the unbound conjugate and the substrate solution was added. After stopping the substrate reaction, the color was measured photometrically. A yellow color indicated the presence of H. pylori antigens in the sample.

According to the present invention, urinary H. pylori antigens can be detected using any suitable detection method. The detection methods include SPR and other biosensor applications, such as thickness shear mode resonator technique, for instance quartz crystal microbalance (QXM), surface acoustic waves (SAW devices) and electrochemical measurements. Surface plasmon resonance (SPR) is specially preferred. The detection method further include immunological methods conventionally used for the detection antigen-antibody complexes, such as EIAs (enzyme immunoassays), ELISAs (Enzyme Linked ImmunoSorbent Assay), fluorescent immunoassays tests or FIAs, turbidometry, nephelometry, competitive tests, time-resolved fluorometry and like (see Immunoassay, Diamandis, E. P. and Christopoulus, T. K., Eds. (1997), AACC Press, USA).

Methods that afford specificity and sensitivity high enough to perform, if desired, a quantitative or a semi-quantitative measurement of the H. pylori antigens are preferred. This is of advantage particularly in the follow-up of the efficacy of the pharmacotherapy of H. pylori infection, whereby the usually quite heavy and long treatment protocol can be changed at an early stage, if the chosen treatment is not effective. Also the total cure can be demonstrated much earlier than with the antibody measurement. Also new or recurrent infections can be easily detected. The quantitative measurement of H. pylori antigens may also provide information on the duration and severity of the infection, which may be helpful in the choice of the medication.

In a spesific embodiment of the invention, freshly collected urine samples are used, whereby the analysis for the presence or absence of Helicobacter pylori is performed subsequent the collection or within a few hours, such as 1 to 4 hours at most. Urine samples that have been stored in a at 4 C for less than two days as well as frozen urine samples can also be used. To ensure the stability of the presence of the H. pylori antigen, fresh samples are preferred.

The present invention is elucidated with the following non-limiting examples.

### EXAMPLE 1

Detection of H. pylori Antigens in a Urine Sample With Surface Plasmon Resonance (SPR)

The presence of Helicobacter pylori antigens in urine was detected with surface plasmon resonance (SPR) using a carrier substrate carrying Fab′-fragments of specific monoclonal anti-H. pylori antibody. The Fab′-fragments were first prepared from a specific monoclonal anti-H. pylori antibody as follows. First, F(ab′)₂ fragments were prepared with ImmunoPure F(ab′)₂ Preparation Kit (PIERCE, USA) from monoclonal anti-H. pylori antibodies, such as monoclonal anti-H. pylori antibodies clones 7101 and 7102 (Medix Biotechnica, Kauniainen, Finland). Other known commercial kits and methods can equally be used. Then the F(ab′)₂ fragments were split into Fab′ fragments with dithiotreitol (DTT, Merck) in a HEPES/EDTA buffer containing 150 mM NaCl, 10 mM HEPES, 5 mM EDTA, pH 6.0, typically over night in a microdialysis tube as described by Ishikawa [Ishikawa, E., J. Immunoassay 4 (1983) 209-320]. Briefly, F(ab′)₂ fragments at a concentration of 0.2-0.5 mg/ml were mixed with HEPES/EDTA buffer and 6.25 mM DTT solution in a microdialysis tube. The dialysis tube was immersed in 250 ml of argon-purged HEPES/EDTA buffer and dialysed over night at room temperature under argon. The Fab′ fragments were maintained under argon and used immediately for attachment.

The solid surface was prepared as follows. Glass slides were first coated with a thin film of titanium to increase the adhesion of gold and then with a thin film of gold by vacuum evaporation. Immediately before use the slides were cleaned in a hot solution of H₂O₂:NH₄:H₂O (1:1:5) and rinsed with water. The slides were attached via an index matching oil to a SPR prism on a Surface Plasmon Resonance Device (SPRDEVI, VTT, Tampere, Finland), the flow cell was assembled on the prism and the flow cell was thoroughly rinsed with a buffer solution containing 10 mM HEPES, 150 mM NaCl, pH 6, prepared in high purity water (18.2 MΩcm; Milli-Q system, Millipore Co., Bedford, USA).

The Fab′-fragments (850 microliters) at a concentration of 70 μg/ml in HEPES/EDTA buffer, pH 6, were added into the flow cell. The Fab′-fragments were allowed to interact with the gold-coated surface typically for 5 minutes, followed by rinsing the surface with the HEPES/EDTA buffer for 5 minutes. Then the buffer was changed to 0.1 M phosphate-buffered saline (PBS), pH 7.2, and 1-1.5 ml of a solution of N-[tris(hydroxy-methyl)methyl]acrylamide at a concentration of 0.15 mg/ml in the PBS buffer were allowed to interact with the surface for 5 minutes. The surface was then blocked with bovine serum albumin (BSA).

The surface was rinsed with PBS, pH 7.2. A negative urine sample (blank) was run at first. Then surface of the carrier substrate was brought into contact with the urine samples to be measured by filling the flow cell of the measuring device for 10 minutes each with the solution to be measured and recording the SPR signal (relative intensity %). The flow cell was rinsed with PBS, pH 7.2, for 5 minutes between measurements.

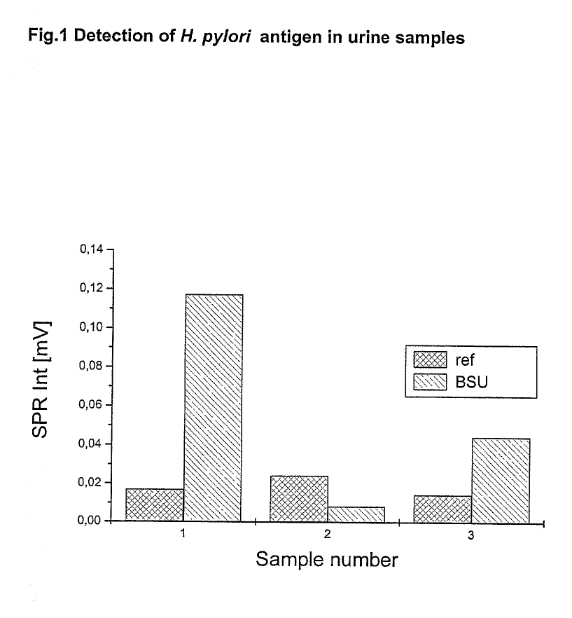

Two of the urine samples measured, samples 1 and 3, were from patients with H. pylori infection verified by biopsy. The third sample, sample 2 was from a patient negative for H. pylori. The results are shown in FIG. 1. The non-specific binding to the layer was 0.0185±0.0050 mV. The response of patient sample 1 was 0.117 mV and of patient sample 3 0.044 mV, 6.3 fold and 2.4 fold, respectively, to that of the background. The negative patient sample 2 gave an intensity of 0.008 mV. The results clearly show that the H. pylori antigen is present in urine samples.

### EXAMPLE 2

Detection of Helicobacter pylori Antigens in Urine Samples by a Sandwich ELISA

The presence of Helicobacter pylori antigens in urine was detected with a sandwich ELISA as follows.

Nunc Maxisorp C8 microtiter plates were coated with monoclonal anti-H. pylori antibodies, clone 7101, (Medix Biochemica, Kauniainen, Finland) at a concentration of 10 μg/ml in 50 mM Tris-HCl, pH 8.0, 100 μl/well, overnight at room temperature (RT). The plates were washed three times with 20 mM PBS, pH 7.4, 0.01% Tween 20 (wash buffer). 250 μl of the blocking solution (20 mM PBS, 1% BSA) were added and the plates were incubated overnight at RT. The plates were washed once with the wash buffer.

Monoclonal antibodies used (1 mg/ml) were labeled with a one-step glutaraldehyde method. Alkaline phosphatase (Sigma P5521, 10 mg/ml, ammonium sulphate suspension, 30 μl, equals 0.3 mg) was sentrifuged at 1200 rpm 15 min. The supernatant was discarded and the enzyme was dissolved in 100 μl of the antibody solution (equals 0.1 mg). The antibody-enzyme-solution was dialyzed overnight at +4° C. against 20 mM PBS, pH 7.4, containing 1 mM MgCl₂ (dialysis buffer). 6 μl of 5% glutaraldehyde was added and the reaction was allowed to proceed for 2 h at RT. 0.5 ml of the dialysis buffer was added and the excess glutaraldehyde was removed by dialysis against the dialysis buffer for 3 h at +4° C. The conjugate was purified with gel filtration on Sepharose CL-6B column eluting with 50 mM Tris-HCl, 1 mM MgCl₂, pH 8.0. The fractions were collected and the AP-activity tested. The fractions with the highest AP-activities were pooled and BSA to a concentration of 1% and Na-azide to a concentration of 0.05% were added. The conjugate was stored at +4° C.

A microtiter plate coated as described above was washed once with the wash buffer. 50 μl of the blank solution (sample dilution buffer), positive and negative controls, and the urine samples were added in duplicate wells. 50 μl of the diluted conjugate (1 to 20 in the sample dilution buffer) were added to each well and the plate was incubated for 60 minutes at RT on a shaker. The wells were washed four times with the wash buffer and 100 μl of pNPP-substrate solution was added to each well. The plate was incubated for 30 minutes at RT on a shaker and the reaction was stopped with 0.45 M NaOH solution. The absorbance was measured at 405 nm (Anthos-reader 2001).

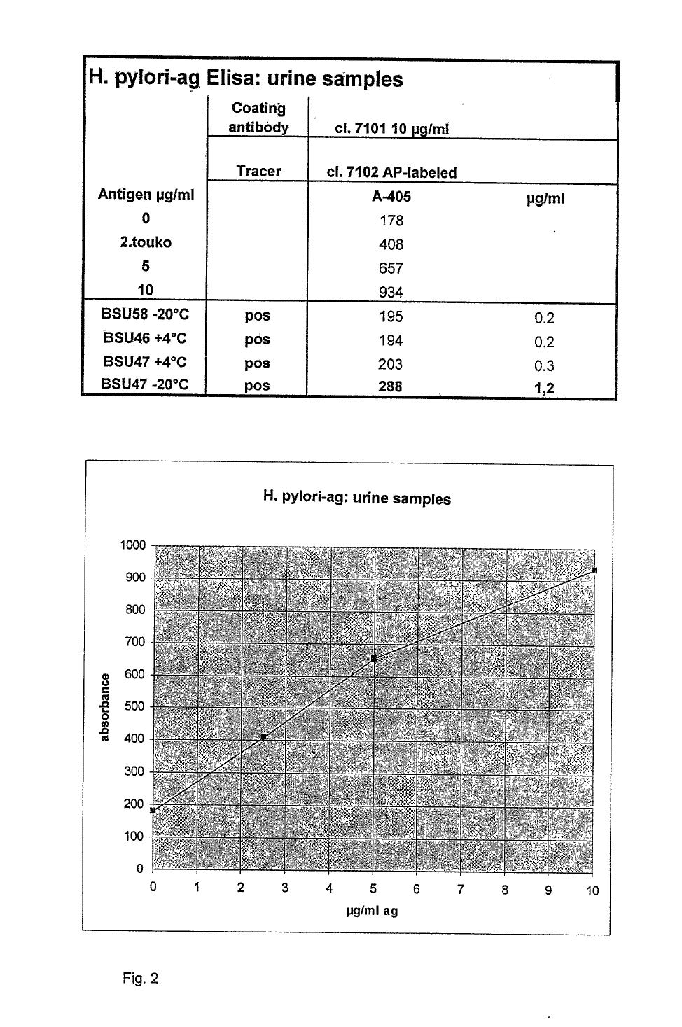

The results, which are shown in FIG. 2, indicate that the H. pylori antigen is present in urine samples.

1. A method of using a urine sample for the detection of the presence or absence of a Helicobacter pylori antigen or a metabolite produced by the bacterium in the diagnosis of Helicobacter pylori infection.

2. A non-invasive method for the diagnosis of Helicobacter pylori infection the method comprising detecting the presence or absence of a Helicobacter pylori antigen or a metabolite produced by the bacterium in a urine sample.

3. The method of claim 2 wherein the presence or absence of a Helicobacter pylori antigen or a metabolite produced by the bacterium in a urine sample is detected by a biosensor-based detection method.

4. The method of claim 3 wherein the presence or absence of a H. pylori antigen or a metabolite produced by the bacterium in a urine sample is detected by serfice plasmon resonance (SPR), thickness shear mode resonator technique, such as quartz crystal microbalance (QXM), surface acoustic waves (SAW devices) and electrochemical measurements.

5. The method of claim 4 wherein the presence or absence of a H. pylori antigen or a metabolite produced by the bacterium in a urine sample is detected by a SPR measurement.

6. The method of claim 2 wherein the presence or absence of a H. pylori antigen or a metabolite produced by the bacterium in a urine sample is detected by an immunological method conventionally used for the detection antigen-antibody complexes, such as EIAs (enzyme immunoassays), ELISAs (Enzyme Linked ImmunoSorbent Assay), fluorescent immunoassays tests or FIAs, turbidometry, nephelometry, competitive tests, time-resolved fluorometry and like.

7. The method of claim 2 wherein the presence or absence of a H. pylori antigen or a metabolite produced by the bacterium in a urine sample is detected by an ELISA method.

8. The method of claim 1 or 2 wherein fresh urine is used.
